(19) United States
(12) Patent Application Publication
(10) Pub. No.: US 2018/0368698 A1
(43) Pub. Date: Dec. 27, 2018

(54) INFORMATION ACQUIRING APPARATUS AND DISPLAY METHOD

(71) Applicant: CANON KABUSHIKI KAISHA, Tokyo (JP)

(72) Inventor: Kenji Oyama, Tokyo (JP)

(21) Appl. No.: 16/064,128

(22) PCT Filed: Feb. 1, 2017
(86) PCT No.: PCT/JP2017/004464

§ 371 Date: Jun. 20, 2018

(30) Foreign Application Priority Data
Feb. 8, 2016 (JP) 2016-021807

(51) Int. Cl. A61B 5/00; A61B 8/08; A61B 6/00

(52) U.S. Cl. CPC A61B 5/0095; A61B 6/5205; A61B 8/5207; A61B 5/0033

(57) ABSTRACT

Provided is an information acquiring apparatus having: an information generating unit generating image data based on signals derived from photoacoustic waves; and a display displaying an image based on the image data, wherein the information generating unit generates first image data based on the signals output from part of a plurality of elements before completing light irradiation, the display displays an image based on the first image data before completing light irradiation, the information generating unit generates second image data based on the signals output from more elements than the part of the plurality of elements, after completing light irradiation, and the display displays an image based on the second image data.

### TECHNICAL FIELD

The present invention relates to an information acquiring apparatus and a display method.

### BACKGROUND ART

One technique to visualize characteristic information of an object is photoacoustic tomography (PAT). PAT is a technique to visualize the functional information of an object using light and an acoustic wave. When a pulsed light (e.g. visible light, near-infrared light) is irradiated to a biological tissue, a light absorbing substance (e.g., hemoglobin in blood) inside a living body absorbs the energy of the pulsed light and momentarily expands and generates an acoustic wave (photoacoustic wave). This phenomenon is called the photoacoustic effect. PAT is a technique to visualize information on the biological tissue by measuring the photoacoustic wave.

By visualizing the optical energy absorption density distribution or absorption coefficient distribution which originated in hemoglobin in a living body, which is acquired by PAT, blood vessels can be imaged. Further, such function information as the oxygen saturation of blood can be acquired using the light wavelength dependency of the generated acoustic wave. Furthermore, PAT, which uses light and acoustic waves, enables minimal invasive image diagnosis, hence burden on a testee can be reduced.

PTL 1 discloses a technique to visualize object information using a probe which includes a plurality of acoustic wave receiving elements disposed at different positions in an approximately spherical space. According to PTL 1, the high sensitivity region can be generated by orienting the high reception sensitivity directions of the plurality of acoustic wave receiving elements toward a predetermined region, and thereby noise of the image can be reduced.

### CITATION LIST

### Patent Literature

PTL 1: U.S. Pat. No. 6,216,025

### SUMMARY OF INVENTION

### Technical Problem

The object information is characteristic information which is acquired by performing image reconstruction on signal data which originated in acoustic waves received by the plurality of acoustic wave receiving elements. For the image reconstruction, back-projection in time domain or Fourier domain, or phased addition processing, or repeated calculation method, which is normally used as a tomographic technique, is used. These processing operations normally require a large calculation amount. Particularly the calculation amount increases if the object information is generated in high definition. Therefore, in the case of generating the object information following the reception of the acoustic wave, while maintaining the image quality as much as possible, the time required for the reconstruction processing must be decreased. In other words, in the case when sequential display to display the image in parallel with the photoacoustic measurement is performed, a problem is how to increase the object information acquisition speed.

Another demand is decreasing the examination time to reduce burden on the testee. To decrease the examination time, it is effective to repeatedly receive the acoustic wave at high-speed. However, if the acoustic wave acquisition time decreases, time that can be spent for the image reconstruction also decreases. As a result, followability to the reception of the acoustic waves for image display drops, which makes sequential display difficult. As described above, increasing the speed of the image reconstruction, which is executed in parallel with the photo-acoustic measurement, is a problem.

The present invention was made with the foregoing in view. It is an object of the present invention to increase followability to the acoustic wave acquisition in the image data generating processing, while maintaining the accuracy of the object information as much as possible in an object information acquiring apparatus.

### Solution to Problem

The present invention provides an information acquiring apparatus, comprising:

an information generating unit configured to generate image data, based on signals acquired by a plurality of elements receiving acoustic waves which is generated from an object by a plurality of times of light irradiation to the object; and

a display controlling unit configured to cause a display unit to display an image based on the image data, wherein

the information generating unit generates first image data using the signals output from part of the plurality of elements before completing the plurality of times of light irradiation,

the display controlling unit causes the display unit to display an image based on the first image data before completing the plurality of times of light irradiation,

the information generating unit generates second image data using the signals output from more elements than the part of the plurality of elements, after completing the plurality of times of light irradiation, and

the display controlling unit causes the display unit to display an image based on the second image data after completing the plurality of times of light irradiation.

The present invention also provides a display method for an image generated based on signals acquired by a plurality of elements receiving an acoustic wave which is generated from an object by a plurality of times of light irradiation to the object, the method comprising:

generating first image data using the signals output from part of the plurality of elements, and displaying an image based on the first image data before completing the plurality of times of light irradiation, and

generating second image data using the signals output from more elements than the part of the plurality of elements, and displaying an image based on the second image data after completing the plurality of times of light irradiation.

### Advantageous Effects of Invention

According to the configuration of the present invention, in the object information acquiring apparatus which uses acoustic waves from the object, followability to the acoustic wave acquisition in the image data generating processing can be increased, while maintaining the accuracy of the object information as much as possible.

Further features of the present invention will become apparent from the following description of exemplary embodiments with reference to the attached drawings.

### BRIEF DESCRIPTION OF DRAWINGS

FIG. 1 is a schematic diagram depicting an apparatus configuration according to Embodiment 1.

FIGS. 2A to 2D are conceptual diagrams depicting the configuration of the probe according to Embodiment 1.

FIG. 3 is a flow chart depicting a flow of object information acquisition according to Embodiment 1.

FIGS. 4A to 4C are schematic diagrams depicting the data structure of the received signals according to Embodiment 1.

FIG. 5 is a schematic diagram depicting another data structure of the received signals according to Embodiment 1.

### DESCRIPTION OF EMBODIMENTS

Embodiments of the present invention will be described with reference to the drawings. Dimensions, materials, shapes, relative positions, and the like, of the elements described below should be appropriately changed depending on the configuration and various conditions of the apparatus to which the present invention is applied. Therefore the scope of the present invention is not limited to the following description.

The present invention relates to a technique to detect an acoustic wave propagated from an object, generate characteristic information inside the object, and acquire the generated information. Therefore the present invention is regarded as an object information acquiring apparatus or a control method thereof, an object information acquiring method and a signal processing method, or a display method. The present invention is also regarded as a program that causes an information processing apparatus, which includes such hardware resources as a CPU and memory, to execute these methods, or a storage medium storing this program.

The object information acquiring apparatus of the present invention includes an apparatus utilizing a photoacoustic effect, which irradiates light (electromagnetic wave) to an object, receives an acoustic wave generated inside the object, and acquires the characteristic information, of the object as image data. In this case, the characteristic information is information on characteristic values corresponding to each of the plurality of positions inside the object, and this information is generated by using the receive signals acquired by receiving the photoacoustic wave.

The characteristic information acquired by the photoacoustic measurement is values reflecting the absorptivity of optical energy. For example, [the characteristic information] includes a generation source of the acoustic wave generated by the light irradiation, an initial sound pressure inside the object, an optical energy absorption density or absorption coefficient derived from the initial sound pressure, and a concentration of a substance constituting the tissue. For the substance concentration, oxygen saturation distribution may be calculated by determining oxygenation concentration and deoxyhemoglobin concentration. Glucose concentration, collagen concentration, melanin concentration, volume fraction of fat or water and the like may be determined.

Based on the characteristic information at each position in the object, a two-dimensional or three-dimensional characteristic information distribution is acquired. The distribution data can be generated as image data. The characteristic information may be determined, not as numeric data, but as distribution information at each position in the object. In other words, such distribution information as the initial sound pressure distribution, energy absorption density distribution, absorption coefficient distribution, and oxygen saturation distribution may be determined. The three-dimensional (or two-dimensional) image data is the distribution of characteristic information on reconstruction units disposed in a three-dimensional (or two-dimensional) space.

The acoustic wave referred to in the present invention is typically an ultrasonic wave, including an elastic wave that is called a sound wave or an acoustic wave. An electric signal converted from an acoustic wave by a transducer or the like is also called an acoustic signal. In this description, the use of the phrase “ultrasonic wave” or “acoustic wave” is not intended to limit the wavelength of the elastic waves. An acoustic wave generated by the photoacoustic effect is also called a photoacoustic wave or a light-induced ultrasonic wave. In electric signal, originating in a photoacoustic wave is also called a photoacoustic signal.

The present invention can also be applied to an apparatus which transmits an acoustic wave to an object, and receives an echo wave reflected inside the object. In this case, the structural information of the object reflecting the change of the acoustic impedance inside the object can be imaged.

### Embodiment 1

### Apparatus Configuration

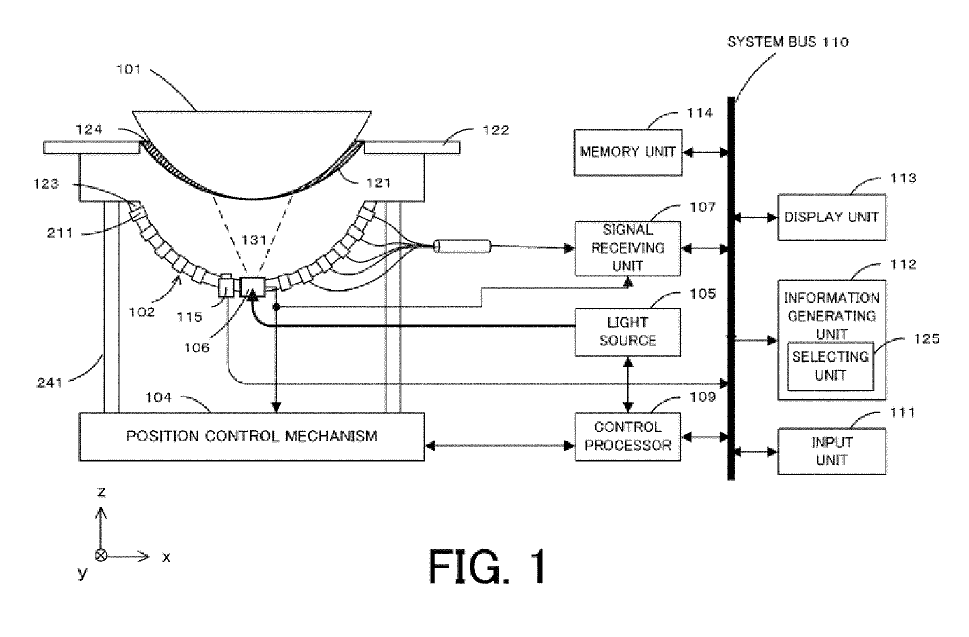

FIG. 1 is a schematic diagram depicting a configuration of an object information acquiring apparatus according to Embodiment 1. This apparatus includes a probe 102 configured to receive a photoacoustic wave which is propagated from an object 101, a position control mechanism 104 configured to control a position of the probe 102, a light source 105, an optical system 106 configured to irradiate light to the object 101, and a signal receiving unit 107 configured to process received signals which were generated by the probe 102.

The apparatus further includes an input unit 111 for the user to operate the apparatus, an information generating unit 112 configured to generate object information based on the received signal, and a display unit 113 configured to display a user interface (UI) for operating the generated object information and the apparatus. The information generating unit functions as the information generating unit and the display controlling unit of the present invention.

The apparatus further includes a control processor 109 which receives various operation instructions of the user via the input unit 111, generates control information that is necessary for generating target object information, and controls each function via a system bus 110. The apparatus further includes a memory unit 114 configured to store acquired photoacoustic signals, generated images, and other information on operations, and an image pickup element 115 configured to image the object 101 in a visible light region.

The object 101 is a measurement target. The measurement target is, for example, a human breast, hand, leg or the like, a living creature other than a human, and a phantom which simulates the characteristic information of a living body, and is used for adjusting the apparatus.

### Element Arrangement in Probe

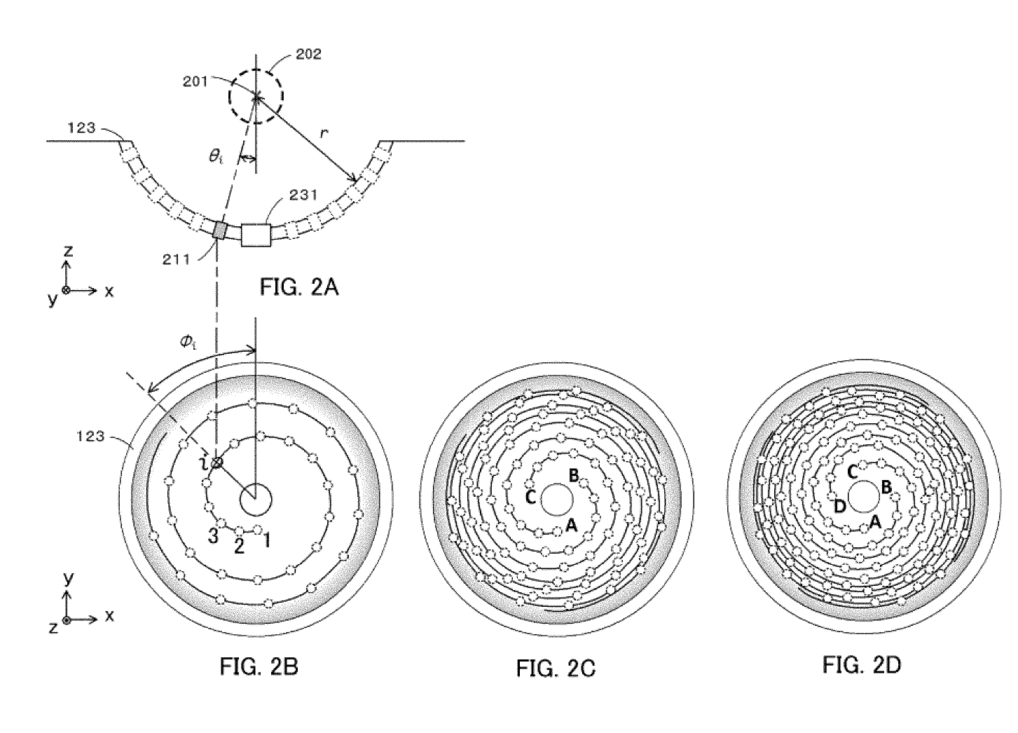

As illustrated in FIG. 2, the probe 102 is constituted by a plurality of acoustic wave receiving elements 211 arranged on a hemispherical supporting unit 123. FIG. 2A is a side view of the probe 102, and FIG. 2B is a top view of the probe 102 in the z axis direction. Each of the plurality of acoustic wave receiving elements 211 detects a photoacoustic wave, which is generated by irradiation of light 131 to the object 101 and propagates from inside the object, and converts the photoacoustic wave into an electric signal. The supporting unit 123 is preferably constituted by a material having a certain degree of strength, such as metal or resin. In the case of filling an acoustic transfer medium inside the supporting unit 123, a container that does not spill the medium is used.

A point 201 in FIG. 2A indicates a curvature center point, which is a mechanical design point of the hemispherical supporting unit 123. Generally each of the plurality of acoustic wave receiving elements 211 has the highest receiving sensitivity in the normal line direction of the receiving plane (surface) thereof, and this direction is also called a directive axis. The acoustic wave receiving element 211 has an effective receiving sensitivity in a predetermined angle range, which is determined on the basis of the directive axis serving as the center. Therefore if the directive axis of each element is concentrated to an area around the curvature center point 201, a high sensitivity region 202 centering around the curvature center point 201 can be formed. The object 101 located in the high sensitivity region 202 can be imaged at high sensitivity and high precision. The high sensitivity region 202 can be defined as a range where an object is imaged at a 50% or higher resolution, compared with the resolution at the curvature center point 201.

The way of arranging the plurality of acoustic wave receiving elements 211 according to the present invention is not limited to the example in FIG. 2. The directive axes of a part or all of the acoustic wave receiving elements may be concentrated to a predetermined region, centering around a mechanical design point, whereby a predetermined high sensitivity region may be formed. The shape of the surface on which the plurality of acoustic wave receiving elements 211 are arranged is, for example, a spherical shape, a hemispherical shape, an open-spherical shape (e.g. spherical crown shape, spherical band shape), a surface with an unevenness on the surface which can be regarded as a sphere, and an ellipsoid which can be regarded as a sphere. If the plurality of acoustic wave receiving elements are arranged along a supporting unit having a spherical crown shape or spherical band shape generated by sectioning a sphere at an arbitrary crass-section, the directive axes are concentrated at the curvature center point of the shape of the supporting unit.

It is preferable that the plurality of acoustic wave receiving elements 211 are arranged in a wide dispersion over the spherical surface of the supporting unit 123 in an approximately uniform manner. In other words, the elements are disposed as isotropically as possible with respect to the high sensitivity region 202. Thereby artifacts caused by the polarization of the measurement points can be suppressed.

The arrangement position of each acoustic wave receiving element 211 is specified in the spherical coordinate system using the radius r, polar angle θ and azimuth angle ϕ with a point 201 on the supporting unit 123 as the origin. This positional information is recorded in advance in the memory unit 114 as the element arrangement data. The information generating unit 112 generates, for the individual acoustic wave receiving element 211, object information by reconstructing an image by associating a received signal and positional information with each other. To reduce artifacts in a high definition display, it is preferable that the entire acoustic wave receiving elements 211 are arranged isotropically from the curvature center point of the supporting unit. Further, in order to maintain the image quality in the sequential display, it is preferable that even in each group, the acoustic wave receiving elements 211 included in the group are arranged isotropically from the curvature center point of the supporting unit.

FIG. 2B illustrates a state in which the plurality of acoustic wave receiving elements are dispersed so that θ and cos (ϕ) are at approximately equal intervals respectively along the spiral route on the spherical surface formed by the supporting unit 123. FIG. 2C and FIG. 2D illustrate the states of additionally disposing one spiral element arrangement indicated in FIG. 2A which is rotated 120° or 90° with respect to the origin. Object information with higher definition can be generated by increasing the number of acoustic wave receiving elements like this. When the acoustic wave receiving elements belonging to one spiral are regarded as one group, the reference signs A, B, C and D in FIG. 2C and FIG. 2D identify each element group. If a plurality of element groups are formed, the element are uniformly dispersed without generating polarization between the element groups.

The arrangement method for the acoustic wave receiving elements is not limited to the above. For example, [the acoustic wave receiving elements] may be arranged such that the Voronoi region in which each element is a kernel point is approximately uniform, or may be arranged such that the distance between adjacent elements is approximately the same, or may be arranged baaed on a Delaunay triangle or Fibonacci lattice. By dispersing the acoustic wave receiving elements 211 to be approximately uniform, accuracy of the object information can be maintained as much as possible, even if visualization target receiving signals are eliminated by selection control.

The present invention can also be applied to an ultrasonic echo apparatus. In the ultrasonic echo apparatus, each acoustic wave transmitting/receiving element 211 may include an acoustic wave transmitting function, or an element for transmitting an acoustic wave may be installed separately. In this case, the object information acquiring apparatus includes an acoustic wave transmitting circuit, and applies driving voltage to each element 211 according to the control information of a control processor 109. In the ultrasonic echo apparatus, a plurality of elements transmit/receive the acoustic wave a plurality of times to/from the object.

An irradiation port 231, to irradiate the light 131 guided from the light source 105 by the optical system 106 to the object 101, is disposed on a bottom surface of the probe 102. The irradiation port 231 may be located at a different location from the probe 102.

For the acoustic wave receiving element 211, it is preferable that the reception sensitivity is high and reception frequency band is wide. For example, an element using piezoelectric ceramics (PST), or a CMUT (capacitive micro-machined ultrasonic transducer) can be used. An MMUT (magnetic MUT) which uses magnetic film, or a PMUT (piezoelectric MUT) which uses piezoelectric thin film can also be used.

(Details of Each Composing Element)

The light source 105 emits pulsed light of which central wavelength is in a near-infrared region. For the light source 105, a solid-state laser (e.g. yttrium-aluminum-garnet laser, titan-sapphire laser) which can emit pulsed light of which central wavelength is in a near-infrared region, is normally used. Other lasers, such as gas laser, dye laser and semiconductor laser, can also be used. Instead of a laser, a light emitting diode or flash map may be used. The light source can irradiate pulsed light to an object for a plurality of times.

To select the wavelength of light in accordance with a light absorbing substance, it is preferable to use a wavelength-variable laser. For example, hemoglobin absorbs light in a 600 to 1000 nm range. The light absorption of water, which constitutes the living body, is at the minimum around approximately 830 nm. Therefore, in a 750 to 850 nm range, light absorption by hemoglobin is relatively high. The absorptivity of light changes depending on the light wavelength when the state of hemoglobin (oxygen saturation) changes. By using this dependency on the light wavelength, functional changes in a living body can be measured. Hemoglobin is a major component of blood vessels, hence a malignant tumor which includes many new blood vessels could be visualized by imaging hemoglobin.

The optical system 106 guides the pulsed light irradiated from the light source 105 toward the object 101, forms a light 131 appropriate for signal acquisition, and emits the light. For the optical system 106, such an optical component as a lens, a prism, a mirror, a diffusion plate and an optical fiber can be used. As a standard on the irradiation of a laser beam, or the like, to the skin, or eyes, the maximum permissible exposure is specified based on such conditions as the wavelength of light, exposure duration and number of repeats of the pulsed light irradiation. The optical system 106 generates light 131 that satisfies this standard.

The optical system 106 includes a detecting mechanism (not illustrated) configured to detect the emission of the light 131 to the object 101, and generate a synchronization signal to receive the photoacoustic wave synchronizing with the detection and control the storage. For example, a part of the pulsed light generated by the light source 105 is split by such an optical system as a half mirror, and guided to the photosensor, whereby the emission of the light 131 can be detected using the detection signal generated by the optical sensor. If a fiber bundle is used for guiding the pulsed light, a part of the fibers may be branched and guided to the photosensor. The generated synchronization signal is input to the signal receiving unit 107 and the position control mechanism 104.

The signal receiving unit 107 is typically constituted by a signal amplifying unit configured to amplify an analog signal received by the probe 102, an A/D converting unit configured to convert an analog signal into a digital signal, and electric circuits such as FPGA and ASIC to control these units. The signal receiving unit 107 collects the received signals from the probe 102 in a time series at a predetermined sampling rate and a predetermined number of samples according to the synchronization signals input from the optical system 106, and converts the received signals into digital signal data.

The control processor 109 operates an operating system (OS) which, for instance, controls and manages the basic resources of program operation, reads program codes stored in the memory unit 114, and executes the functions of the embodiment to be described later. The control processor 109 also manages the object information acquiring operation, upon receiving event notifications, which are generated by the user who performs various operations (e.g. measurement start) via the input unit 111. The control processor 109 also controls each hardware component via the system bus 110.

The position control mechanism 104 changes the relative positions between the probe 102 and the object 101. Thereby the high sensitivity region 202 moves inside the object, and high definition object information can be acquired in a wide range. The position control mechanism 104 is constituted by a driving unit, such as a motor, and

a driving mechanism, such as a lead screw mechanism, a link mechanism, a gear mechanism and a hydraulic mechanism:, which transfers this driving force. The position control mechanism 104 controls the positions of the pulsed light 131 and the probe 102 according to the scanning control information from the control processor 109. The position control mechanism 104 also includes an optical or a magnetic encoder, or the like, to acquire position control information, and acquires the position control information when signals are received, in accordance with the synchronization signal of the irradiation of the pulsed light 131, which is input from the optical system 106. The position control mechanism corresponds to the position controlling unit of the present invention.

The input unit 111 is an input apparatus to set parameters on the object information to be generated, instruct the start of measurement, set an observation parameters for the generated object information, and perform image processing operation on an image, for example. Generally the input unit is constituted by a mouse, keyboard, touch panel or the like, and according to the user operation, notifies events to the OS and other software executed by the control processor 109. In the case of using a touch panel for the input unit 111, the display unit 113 has this input function.

The information generating unit 112 reconstructs the image for signal data originated in the electric signals output by the plurality of acoustic wave receiving elements 211, and generates the image data indicating the tissue information inside the object. For the image reconstruction, a known method (e.g. back-projection in time domain or Fourier domain, inverse problem analysis in repeated phased addition processing,) can be used. The information generating unit 112 is normally constituted by a GPU (graphics processing unit), for example, which has high performance calculating functions and graphic display functions. By increasing the performance of the information generating unit 112, time required for generating image data can be decreased.

The information generating unit 112 further includes a selecting unit 125 configured to select target signal data to generate object information, out of the signal data stored in the memory unit 114. The selecting unit 125 may be a physical circuit or may be configured as a program module. By the selecting unit 125 controlling the selection of the received signals to be used for image reconstruction, the followability to the object information generation improves while maintaining the accuracy of the object information to be generated as high as possible. As a result, image data can be generated during one cycle of the apparatus repeating signal acquisition, or during one refresh rate cycle in the moving image display. If the received signals are skipped in the acoustic wave receiving element units the calculation amount per voxel can be eliminated for the amount of skipped elements, and a major processing time reduction effect is implemented.

It is preferable that the signal receiving unit 107 holds the data of the received signals in continuous storage areas of the volatile memory of the memory unit 114. Thereby the information generating unit 112 can sequentially access the data during the image reconstruction. In some cases, it is advantageous for the information generating unit 112 if the data size of the processing target is a power of 2. Therefore it is preferable to set a number of acoustic wave receiving elements 211, a sampling frequency or the like so that the size of the total received signal data becomes a power of 2.

The display unit 113 displays an image and numeric data of the object information generated by the information generating unit 112. The display unit 113 may display a UI to operate an image and apparatus. For the display emit 113, a liquid crystal display, an organic EL (electro luminescence), a plasma display, a field emission display or the like can be used.

The information generating unit 112 generates object information in accordance with such display formats as a moving image display, an integration display and a comparative display. In the moving image display, the object information to be displayed is successively updated based on a plurality of received signals which are successively acquired. To observe the time-based change of the object 101 in the moving image display, it is preferable that the processing from the signal acquisition to the display of the object information can follow the repeat cycle of the signal acquisition or the refresh rate of the moving image display. Furthermore, it is preferable that real-time operation can be implemented within the time constraints.

In the integration display, S/N is improved by integrating the object information based on a plurality of signal acquisitions. Further, if object information, which is generated and integrated based on signals acquired at a plurality of positions, is displayed, the scanning process can be visually recognized, and object information in a wide range can be generated and displayed. In the comparative display, object information generated based on received signals, acquired under a plurality of different conditions, is displayed on the same screen side by side. Thereby comparative observation can be supported for the user. For example, when the signal acquisition is repeated while changing the light wavelength, dependence of the object 101 on the light wavelength can be more easily observed by displaying the object information acquired at each light wavelength side by side. The method for displaying the optical acoustic image is arbitrary, and for example, one arbitrary cross-sectional image, a maximum value projected image in an arbitrary viewing direction and at an arbitrary slab thickness, or a three-dimensional volume image can be used.

The memory unit 114 is constituted by a volatile or non-volatile memory that is required for operating the control processor 100. The volatile memory is used for temporarily holding data. The non-volatile memory, such as a hard dish, stores and holds acquired signal data, generated image data, arrangement data of the acoustic wave receiving elements 211, related numeric data, diagnostic information, software program codes and the like.

The image pickup element 115 images the object 101 and outputs the image signals thereof. For the image pick/up element 115, an optical image pickup element, such as a CCD sensor and a CMOS sensor, is typically used. The user can specify, for instance, the signal acquisition positions and the range thereof, required for generating target object information, on the image captured by the image pickup element 115.

To match acoustic impedance, it is preferable to dispose an acoustic transfer medium 124, such as water, oil and gel for ultrasonic measurement in the space between the object 101 and a holding unit 121 thereof, so that no gap is generated in a space. It is also preferable that the space between the holding unit 121 and the supporting unit 123 of the probe 102, which is a photoacoustic wave propagation path, is filled with a medium having a high acoustic wave propagation efficiency. Further, this medium is preferably transparent with respect to the light 131, since this propagation path is also a propagation path of the light 131. The holding unit 121 is not always necessary, but has an effect to maintain the shape of the object 101 and stabilize the measurement, and also to make the light quantity calculation easier. The holding unit 121 preferably has high transmittance with respect to light and an acoustic wave.

### Processing Flow

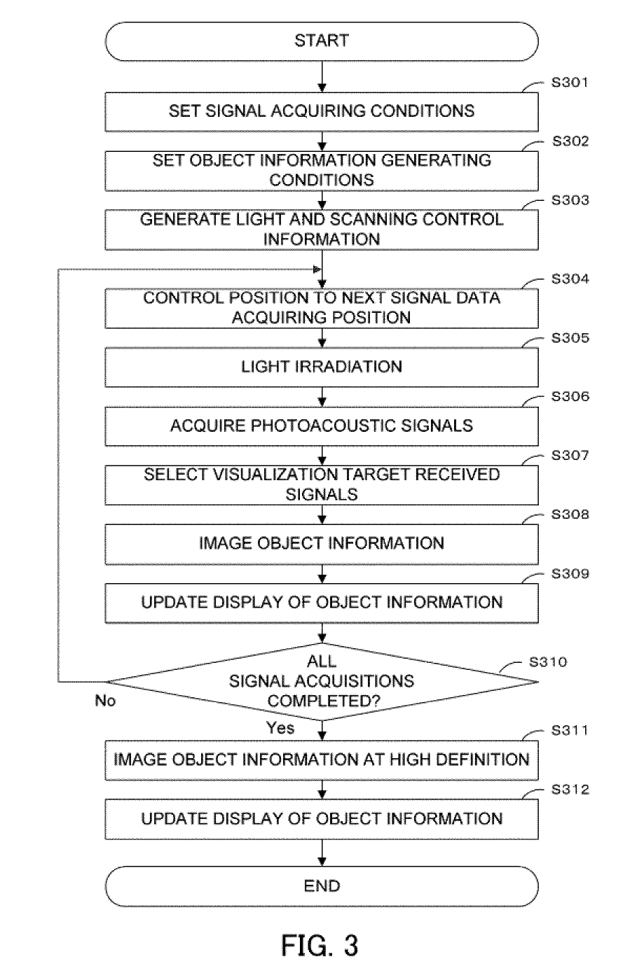

The flow of the object information acquisition according to Embodiment 1 will be described next with reference to FIG. 3. In step S301, the control processor 109 sets the signal acquiring conditions according to the specification by the user via the input unit 111. To set the signal acquisition positions and acquire signals in a wide range, the user specifies the scanning region, light wavelength to be used for measurement, repeat frequency of signal acquisition and the like. The repeat frequency of the signal acquisition corresponds to the repeat frequency of light irradiation by the light source 105 in this embodiment, and corresponds to the repeat frequency of the ultrasonic transmission if an ultrasonic wave, not light, is transmitted. A plurality of signal acquisition settings may be stored in the memory unit 114 as predetermined values, and the user may select a desired setting therefrom.

In step S302, the control processor 109 sets the object information generating conditions according to the specification by the user via the input unit 111. The user specifies size, resolution and the like of the object information to be generated. The display format of the object information can also be specified. For the display format, a moving image display to successively update the object information, an integration display of object information which is successively generated, and a comparative display at a plurality of light wavelengths, for example, can be specified. Besides the repeat frequency of signal acquisition, the refresh rate for a display can be set as well.

From the settings of required sizes, resolutions or the like, the image reconstruction time to generate the object information can be calculated. Further, from the repeat frequency of signal acquisition or the refresh rate of the display, the time constraints to generate the object information can be calculated. Furthermore, a received signal selection amount that is required for following the repeat frequency of signal acquisition or the refresh rate of the display can be estimated,

In step S303, the control processor 109 generates control information of light and scanning in accordance with the conditions which were set in the steps thus far. In concrete terms, the signal acquisition position, scanning path, scanning speed, scanning density, acceleration/deceleration profile during scanning, number of times of light irradiation, repeat frequency of light irradiation and the like are generated. If a plurality of light wavelengths are used, control information on switching the light wavelength is also generated. The control processor 109 outputs the generated control information to the position control mechanism 104, light source 105 and signal receiving unit 107.

The control processor 109 also sets selection control of the received signals, which the selecting unit 125 selects as the visualization targets, based on the estimation of the selection amount of the received signals to be the visualization targets. The range of possible values of each of the above mentioned conditions is limited depending on the configuration of the apparatus. Therefore a control table to select the visualization target received signals may be stored in the memory unit 114 in advance. In this case, the user selects the signals using the input unit 111.

In step S304, the position control mechanism 104 moves the probe 102 to the next photoacoustic signal acquiring position according to the position control information.

In step S305, the light source 105 generates the pulsed light according to the control information, such as the light wavelength and repeat frequency of light irradiation. The pulsed light emitted from the light source 105 is shaped to the light 131 via the optical system 106, and is irradiated to the object 10c1. If a plurality of light wavelengths are used, the light wavelength switching control is also performed. When the irradiation of the light 131 is detected, the optical system 106 generates a synchronization signal and sends the synchronization signal to the position control mechanism 104 and the signal receiving unit 107. In the case of the ultrasonic echo apparatus, the ultrasonic wave is transmitted in this step.

In step S306, the probe 102 detects the photoacoustic wave generated from the object 101, and the signal receiving unit 107 starts receiving the photoacoustic signal synchronizing with the synchronization signal, which is input from the optical system 106. The received signal data is held in the memory unit 114 via the system bus. The position control mechanism 104 acquires the position control information when the light 131 is irradiated, synchronizing with the synchronization signal that is input from the optical system 106. The memory unit 114 associates the received signal data with the position control information, and holds this information.

### Storage and Selection of Received Signal Data

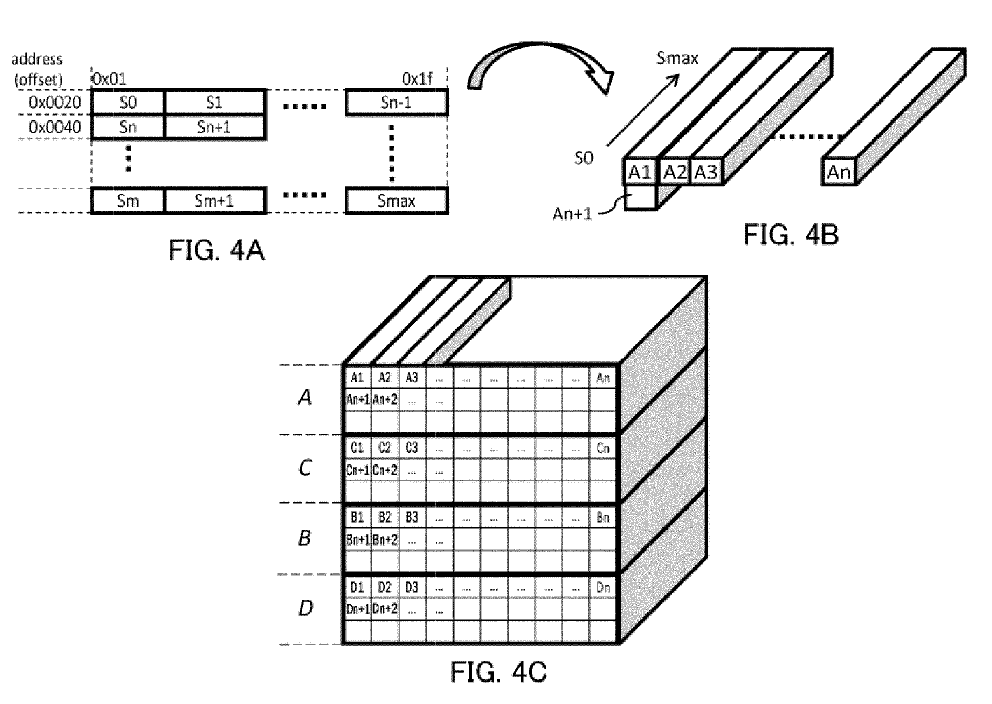

In step S307, the selecting unit 125 selects the received signals to be the visualization targets. The received signal selection control according to this embodiment will be described with reference to FIG. 4. FIG. 4 illustrates the general structure of the received signal data which the signal receiving unit 107 outputs based on the arrangement of the acoustic wave receiving elements 211 in FIG. 2D.

FIG. 4A illustrates a data format of the received signal generated by an element A1, which is one of n number of acoustic wave receiving elements (A1, A2, A3, . . . An). The data is stored in continuous regions starting with an address specified in the operation of the storage region. A signal data group (S0, S1, . . . , Sn, . . . , Sm, . . . , Smax) is a data group which was collected by one element in a time series, and each data included in the data group corresponds to one sample respectively.

FIG. 4B illustrates received data of each acoustic wave receiving element 211, integrated and schematically expressed as one rectangular parallelepiped. Then next to the signal data of the acoustic wave receiving element A1 depicted in FIG. 4A, the signal data of the elements A2 to An is continuously disposed in the storage region. The data of the element An+1 and later may also be continuously disposed.

FIG. 4C illustrates a data structure of received signals which are acquired by one signal acquisition according to this embodiment. This kind of data is stored in continuous storage regions in the memory unit 114. Here the received signal data belonging to one spiral forms one data block. After storing the data blocks in group A, the signal data of group C, group B and group D are stored in continuous regions.

By storing signal data for each group like this, the selecting unit 125 can select the visualization target received signals in group units. For example, selecting only one group, selecting two groups, or selecting three groups is possible. When a group is selected, artifacts can be suppressed if the measurement points of the visualization target received signals do not become polarized. For example, if two groups are selected in this example, a combination of groups “A and C” or groups “B and D” is selected. The data groups are held in FIG. 4C in the sequence of group A, C, B and D, because when the above mentioned selection of “A and C” or “B and D” is used, sequential data access becomes possible, and processing time can be decreased. The arrangement of the data groups in a storage region, however, is not limited to this.

The selecting unit 125 selects visualization target received signals in group units. If the display format is the update display or integration display, it is preferable that the selecting unit 125 changes the visualization target groups between the first signal acquisition and the subsequent signal acquisition. For example, if only one group is selected as the visualization target, the groups are selected in the sequence of A→B→C→D every time display is updated. If two groups are selected, the selection patterns of “A and C”→“B and D” are repeated alternately. Thereby the acoustic wave receiving elements are net polarized in a specific direction when the image data is generated, and isotropic properties increase. In other words, the selecting unit 125 selects signals output from part of a plurality of acoustic wave receiving elements for sequential display. In the sequential display, the first image data is generated using electric signals corresponding to part of a plurality of times of light irradiation.

In the case of comparatively displaying each object information generated with different light wavelengths, it is preferable to generate object information in the same way using the first wavelength and second wavelength. For example, if the selecting unit 125 selects group A to generate the object information with the first wavelength, the selecting unit 125 also selects group A with the second wavelength. If the selecting unit 125 changes to group C to generate the object information with the first wavelength, the selecting unit 125 also selects group C with the second wavelength.

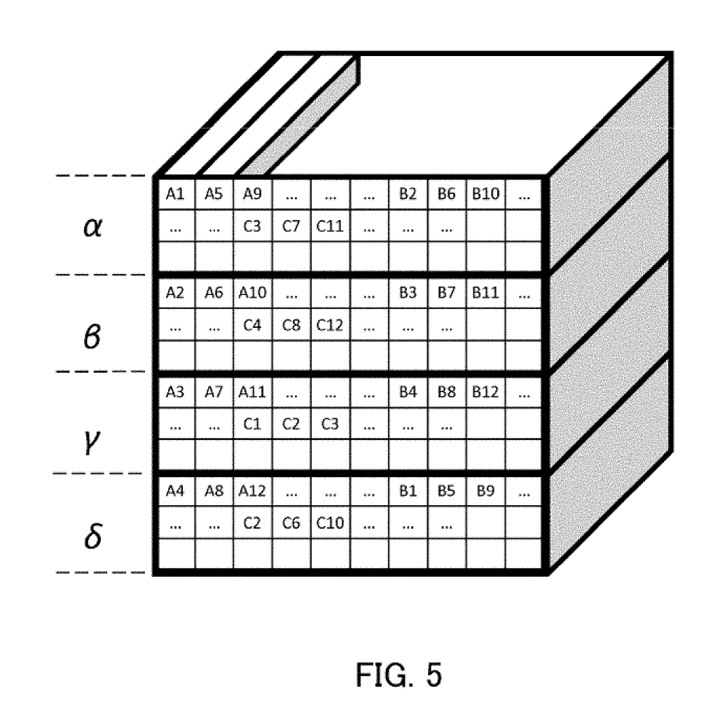

FIG. 5 illustrates another example of a data structure. In the date structure in FIG. 4, elements are grouped for each spiral. However, in FIG. 5, they are grouped for a certain number of elements, not taking into account spirals. In this data structure as well, the visualization target received signals can be appropriately selected, similarly to FIG. 4. Further, the arrangement of elements belonging to each group becomes isotropic. The selecting unit 125 selects the visualization target received signals in the units of α, β, γ and σ.

Description continues referring back to the flow chart. In step S308, the information generating unit 112 reconstructs an image using the received signals selected in step S307. If the image reconstruction speed delays the repeat cycle of signal acquisitions, signal data that is successively acquired is managed in queues. In step S309, the display unit 113 updates the display using the object information generated in step S308. Thereby a sequential display following one photoacoustic measurement completes. In this description, the sequential, display, in which the first image data is generated, is also called the first display. The first display is an image display before completing a plurality of times of pulsed light irradiation. As mentioned above, in the first display, electric signals which originated in the selected partial elements are used for generating the first image data.

In step S310, it is determined whether all the signal acquisitions completed. For example, this determination is performed based on whether scanning of the object has completed, or whether a predetermined time has elapsed. If the signal acquisition is not completed, processing returns to step S304, and the probe performs the photoacoustic measurement at the next position. If the signal acquisition is completed, processing moves to step S311.

In step S311, the information generating unit 112 generates image data also using received signals which were not used in each step of sequential display. In this data generation for a high definition display, all the data need not be used. In the case of the data generation for the high definition display, received signals output from more elements than the partial elements out of the plurality of acoustic wave receiving elements, which were used for generation of one item of data for sequential display, can be used. In other words, in the high definition display, a second image data is generated using electric signals corresponding to light irradiation more than the part of a plurality of times of light irradiation used for the first image data. In step S312, the display unit 113 updates the display with the object information generated in step S311. Thereby high definition display is performed based on more received signals compared with step S309. Steps S311 and S312 may be executed not immediately after examination but on another occasion.

In this description, the high definition display, in which second image data is generated, is also called the second display. The second display is an image display after completing a plurality of times of pulsed light irradiation. In the control of the present invention, the first display and second display are switchable. By using, in the second display, the electric signals output from more elements than the partial elements selected in the first display, a higher definition image can be generated.

According to this embodiment, a plurality of acoustic wave receiving elements are disposed isotropically at different positions on a curved surface of the probe, and the elements are divided into a plurality of groups. Further, the elements included in each acoustic wave receiving element group are disposed as uniformly as possible. Then the selecting unit selects the received signals in group units. Thereby the elements can be isotropically selected with respect to the high sensitivity region, regardless which group is selected. As a result, sequential display having high followability to the photoacoustic measurement can be implemented, while maintaining the accuracy of the object information as high as possible. Further, after the scanning and photoacoustic measurement end, image data suitable for high definition display is generated by image reconstruction, which uses the data output from more elements than the case of each sequential display.

### Embodiment 2

In Embodiment 1, the information generating unit 112 has the functions of the selecting unit 125. In this embodiment, the signal, receiving unit 10 has the functions of the selecting unit 125. In other words, the signal receiving unit 107 performs the selection control to transfer a part or all of the received signals to the system bus 110. Instead of controlling whether a transfer is performed or not, the priority ranking to transfer the received signals to the system bus 110 may be controlled. In this case, the visualization target received signals are transferred with priority.

According to this embodiment, the transmission amount from the signal receiving unit 107 to the system bus 110 can be reduced. As a result, the transmission time decreases, and the followability performance in the object information display further improves. It is also preferable that the signal receiving unit 107 generates one composite signal by adding up the received signals of a plurality of neighboring acoustic wave receiving elements, so as to reduce the amount of received signals. As the number of acoustic wave receiving elements to be added up is higher, the effect of reducing the transmission amount is larger. On the other hand, accuracy of the object information to be generated further drops, since the unique information based on individual positions of the acoustic wave receiving elements that are added up is lost.

### Embodiment 3

In this embodiment, a configuration to improve image quality in the sequential display will be described. The input unit 111 of this embodiment receives the specification of a region of interest, which is the target of the image reconstruction, from the user. The region of interest, which is set inside the object 101, is a predetermined range of which the user particularly desires visualization. The specification is received, for example, via the numerical input in the coordinate system, the range input using a mouse or touch pen, or the selection from a plurality of candidates which are set in advance. The control processor 100 may automatically set the region of interest in accordance with the conditions on the objects (e.g. shape, size), measurement time, knowledge acquired by other modalities and the like. The position control mechanism 104 may control the scanning range according to the region of interest. Measurement time can be decreased by decreasing the number of acoustic wave acquiring positions, compared with the case of imaging the entire object 101.

The selecting unit 125 of this embodiment selects predetermined signals that are used for sequential display and high resolution display respectively, according to the region of interest which has been set. The standard to select the electric signals is the positional relationship between the arrangement positions of the elements which output the electric signals and the region of interest. In other words, in this embodiment, elements, which are dispersed to be approximately uniform with respect to the region of interest, are selected when the image data of the region of interest is generated. In other words, each element included in the element group is disposed isotropically with respect to the region of interest.

If the candidates of the region of interest have been set in advance, the element groups can be set in advance as well. According to this embodiment, the data amount to be the base of the image reconstruction can be reduced, and processing time can be decreased while maintaining the image quality in the region of interest, particularly in the sequential display.

### Other Embodiments

The object of the present invention can also be implemented by the following. In other words, a storage medium (or recording medium) storing program codes of software which implement the above mentioned functions of the embodiments are supplied to a system or an apparatus. Then, a computer (or CPU or MPU) of the system or apparatus reads the program codes stored in the storage medium, and executes the program. In this case, the program codes which are read from the storage medium implement the functions of the embodiments, and the storage medium storing the program codes constitute the present invention. The storage medium may be non-transitory.

When the computer executes the program codes which were read, an operating system (OS) or the like running on the computer performs a part or all of the actual processing based on the instructions of the program codes. The present invention includes the case of implementing the above mentioned functions of the embodiments by this processing.

Further, it is assumed that the program codes read from the storage medium are written in a memory of a function expansion card inserted into the computer slot, or a memory of a function expansion unit connected to the computer. The case of, for instance, the function expansion card or CPU of the function expansion unit performing a part or all of the actual processing based on the instructions of the program codes, and implementing the above mentioned functions of the embodiments by this processing, is also included. When the present invention is applied to the storage medium, the program codes corresponding to the above described flow chart are stored in the storage medium.

Persons skilled in the art can easily construct a new system appropriately combining various technique according to each of the above embodiments, and such a system implemented by various combinations is also within the scope of the present invention.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium, may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-21807, filed on Feb. 8, 2016, which is hereby incorporated by reference herein in its entirety.

1. An information acquiring apparatus, comprising:
an information generating unit configured to generate image data based on signals acquired by a plurality of elements receiving acoustic waves which is generated from an object by a plurality of times of light irradiation to the object; and
a display controlling unit configured to cause a display unit to display an image based on the image data, wherein
the information generating unit generates first image data based on the signals output from part of the plurality of elements before completing the plurality of times of light irradiation,
the display controlling unit causes the display unit to display an image based on the first image data before completing the plurality of times of light irradiation,
the information generating unit generates second image data based on the signals output from more elements than the part of the plurality of elements, after completing the plurality of times of light irradiation, and
the display controlling unit causes the display unit to display an image based on the second image data after completing the plurality of times of light irradiation.

2. The information acquiring apparatus according to claim 1, further comprising:
a memory unit configured to store the signal after associating the same with the element to which the signal is output; and
a selecting unit configured to select from the signals stored in the storing unit a signal output from a predetermined element, wherein
the information generating unit generates the image data based on the signals selected by the selecting unit.

3. The information acquiring apparatus according to claim 2, wherein
the plurality of elements are divided into a plurality of groups, and
the selecting unit selects part of the plurality of groups to generate the first image data so as to select the signal corresponding to the element included in the selected partial groups.

4. The information acquiring apparatus according to claim 3, wherein
the plurality of elements are disposed so that directional axes of the plurality of elements concentrate.

5. The information acquiring apparatus according to claim 4, wherein
the plurality of elements are divided into the plurality of groups, so that the plurality of
elements included in each of the groups are dispersed in an approximately uniform manner with respect to a region where the directional axes concentrate.

6. The information acquiring apparatus according to claim 3, wherein
the plurality of elements are divided into the plurality of groups, so that the plurality of elements included in each of the plurality of groups are isotropically disposed.

7. The information acquiring apparatus according to claim 3, wherein
the plurality of elements are supported by a hemispherical or spherical crown-shaped support unit, and
in each of the plurality of groups, the plurality of elements included in the group are dispersed in an approximately uniform manner from the center of curvature of the supporting unit.

8. The information acquiring apparatus according to claim 3, wherein
the memory unit stores the signals in continuous storage regions for each set of the plurality of groups.

9. The information acquiring apparatus according to claim 2, wherein
the plurality of elements are disposed so as to be a plurality of spirals, and
the selecting unit selects the signals to generate the first image data by selecting part of the plurality of spirals.

10. The information acquiring apparatus according to claim 1, further comprising a position controlling unit configured to change relative positions of the plurality of elements and the object, wherein
the first image data is displayed when the position controlling unit is performing the control.

11. The information acquiring apparatus according to claim 1, wherein
the information generating unit generates the first image data for each light irradiation, based on the signals output from the partial elements, and
the display controlling unit causes the display unit to display an image based on the image data, for each light irradiation, as a display of the image data.

12. The information acquiring apparatus according to claim 1, wherein
the information generating unit generates the image data which indicates information on at least one of a generation source of the acoustic wave, initial sound pressure of the acoustic wave, optical energy absorption density, absorption coefficient, and concentration of a substance constituting the object.

13. The information acquiring apparatus according to claim 1, wherein
as the first image data, the information generation unit generates image data that indicates initial sound pressure distribution or optical energy absorption density distribution, and as the second image data, the information generation unit generates image data that indicates absorption coefficient distribution, or concentration distribution of a substance constituting the object.

14. The information acquiring apparatus according to claim 1, further comprising:
a light source configured to perform the plurality of times of light irradiation; and
the plurality of elements.

15. A display method for an image generated based on signals acquired by a plurality of elements receiving an acoustic wave which is generated from an object by a plurality of times of light irradiation to the object,
the method comprising:
generating first image data based on the signals output from part of the plurality of elements, and displaying an image based on the first image data before completing the plurality of times of light irradiation, and
generating second image data based on the signals output from more elements than the part of the plurality of elements, and displaying an image based on the second image data after completing the plurality of times of light irradiation.

16. A non-transitory storage medium which stores a program causing a computer to execute the display method according to claim 15.
